(19) United States
(12) Patent Application Publication
(10) Pub. No.: US 2009/0087495 A1
(43) Pub. Date: Apr. 2, 2009

(54) Food for Improving Motor Function

(75) Inventors: Koji Toyomura, Ibaraki (JP); Mikako Sato, Ibaraki (JP); Jun Ichikawa, Ibaraki (JP)

Correspondence Address: BIRCH STEWART KOLASCH & BIRCH, PO BOX 747, FALLS CHURCH, VA, 22040-0747, US

(73) Assignee: Nippon Meat Packers, Inc., Osaka-shi, Osaka (JP)

(21) Appl. No.: 11/920,573

(22) PCT Filed: May 19, 2005
(86) PCT No.: PCT/JP2005/009605

§ 371 Date: Aug. 6, 2008

(51) Int. Cl. A61K 35/12; A61K 38/05; A61K 33/26; A61K 36/31

(52) U.S. Cl. 424/520; 514/19; 424/646; 424/755

(57) ABSTRACT

A food for improving motor function comprising as effective components carnosine and/or anserine and a material for improving motor performance (a material for motor energy source, a material for promoting conversion into energy and/or an antioxidant) is provided. In the invention, by synergistic effect of these components, the motor function or stamina can be improved and recovery from fatigue can be promoted in humans and mammals. Further, any of the effective components of the food for improving motor function of the invention is a naturally occurring component, therefore, the food has a characteristic of high safety.

### TECHNICAL FIELD

This invention relates to a food for improving motor function. More particularly, it relates to a functional food capable of improving the exercise ability and stamina and promoting recovery from fatigue of humans and mammals.

### BACKGROUND ART

Recently, with increase in people's desire for better health, sports for keeping health and improving physical strength are becoming more and more popular. Doing proper aerobics is said to be effective for preventing obesity and hypertension and for preventing life-style related diseases, and it is recommended to do running and walking for the purpose of maintaining and promoting health.

Further, higher level training at athletic clubs or training gymnasiums is becoming increasingly popular. Following foods for people doing physical training or sports are presented:
1) foods for supplying sugar as energy source for exercise, and mineral and water that will be consumed during the exercise;
2) foods having function such as improvement of stamina and recovery from fatigue. These foods are presented in various forms such as powder, tablets and drinks.

However, such conventional foods having a function such as improvement of stamina and recovery from fatigue are not enough to meet the demand, and there has been a demand for foods having stronger effects.

In order to solve such a problem, the inventors have intensively studied, and found when carnosine and/or anserine are used with a material that is said to have a function of improving exercise ability (hereinafter called a material for improving motor performance), it is possible to improve the exercise ability and stamina, and to enhance remarkably the recovery from fatigue by synergistic effect of carnosine and/or anserine and the material for improving motor performance.

The present invention is based upon the knowledge thus obtained, and the object of the invention is to provide food for improving motor function comprising carnosine and/or anserine and the material for improving motor performance.

It is known that carnosine and anserine have anti-stress effects (Japanese-Laid Open Patent 1997-20660), and also improve exercise ability (Japanese Laid-Open Patent 2002-51730). However, it is not known that combination use of carnosine and/or anserine and the material for improving motor performance brings effect of enhancing the physical performance and stamina and effect of recovering from fatigue by the synergistic effect of these components.

### DISCLOSURE OF THE INVENTION

The present invention provides a food for improving motor function containing carnosine and/or anserine, and the material for improving motor performance.

The food preferably contains a material for motor energy source (creatine, amino acid, whey protein and/or soybean protein), a material for promoting conversion into energy (caffeine, citric acid, carnitine, thioctic acid and/or iron compound) and/or an antioxidant (polyphenols, ubiquinone (CoQ10), tocotrienol, liquamen, maca extract powder, hercampure extract powder and/or banasulin (trade name, banaba leaf extract)) as the material for improving motor performance.

Also, the food preferably contains carnosine and/or anserine, and the material for improving motor performance at a ratio of 1:0.5 to 10 (by weight), and further, the food preferably contains carnosine and/or anserine in a form of chicken extract or its powder.

Incidentally, the food of the invention is a concept including feed.

### BEST MODE CARRYING OUT THE INVENTION

As described above, the present invention is the food for improving motor function containing carnosine and/or anserine, and the material for improving motor performance.

Carnosine and anserine as effective components of the invention are known substances. In the invention, carnosine and anserine can be used individually or in combination. Carnosine and anserine can be used in a form of free and an acid-addition salt (e.g. chloride, acetate, citrate, etc.) or an alkali-addition salt (e.g. sodium salt, potassium salt, ammonium salt, etc.).

Carnosine and anserine prepared by chemical synthesis and fermentation can be used, but preferably it is advantageous to use extract of natural product in cost. That is, since carnosine and anserine are much contained in chicken and fish, they can be obtained by using waste materials generated in the manufacture of processed food using these materials.

Carnosine and anserine derived from chicken are preferable in particular, which can be obtained from chicken in accordance with the method described in Japanese Laid-Open Patent 2002-51730. One of the examples is specifically explained. That is, chicken is sliced, and after adding hot water and acid or alkali, the solution obtained by extracting the chicken in hot water is desalted in a column or the like and treated with protease.

In this case, any kind of acid can be used provided that it can be added to food, and hydrochloric acid, citric acid, acetic acid and ascorbic acid can be exemplified as an acid substance, and it is preferable to use citric acid.

Also, when the extraction is carried out in hot water after adding alkali, sodium hydroxide, potassium hydroxide, triethanol amine, etc. can be exemplified as an alkaline substance.

The amount of acid or alkali used as against chicken is 0.01% by weight or over, usually about 0.1 to 2.0% by weight.

There is no particular limitation on the condition for extraction, but usually it is treated at 90° C. for about four hours. Also, the extracted liquid can be desalted by a conventional method.

As to protease used after desalting, any kind of enzyme capable of decomposing protein can be used. Papain, trypsin, and pepsin can be exemplified as protease, and it is preferable to use papain. Two or more kinds of such protease can be used in combination. The amount of protease used as against the extract of chicken is 0.005% by weight or over, usually about 0.01 to 10% by weight. There is no particular limitation on the condition for treating enzyme, but it is usually treated at 50° C. for about one hour. After enzyme treatment, the solution is heated for the purpose of deactivation. The resultant solution is neutralized, purified and desalted by a column or the like, and then dried by various methods, for example, hot air drying, spray drying and freeze drying to give a processed chicken powder (hereinafter called chicken extract powder).

The chicken extract powder thus obtained is a mixture of amino acid and peptide containing carnosine and anserine, preferably containing carnosine by 3 to 20%, anserine by 5 to 30%, and more preferably containing carnosine by 3 to 20%, anserine by 10 to 30%.

The material for improving motor performance that is a kind of effective component of the invention is a material in general intended to improve the exercise ability. The material for improving motor performance may include the material for motor energy sources that has a function as energy sources during exercise (e.g. creatine, amino acid, whey protein, soybean protein, etc.), the material for promoting conversion into energy that has a function to promote converting energy sources such as glucose and fat into energy (e.g. caffeine, citric acid, carnitine, thioctic acid, iron compound, etc.) and the antioxidant having an anti-oxidation function for reducing active oxygen generated due to exercise and causing lesion to muscle cells (e.g. polyphenols, ubiquinone (CoQ10), tocotrienol, liquamen, maca extract powder, hercampure extract powder, banasuline, etc.).

One, two or more kinds of the above-mentioned material for motor energy sources, material for promoting conversion into energy and antioxidant may be used.

In the components of the above-mentioned material for improving motor performance, essential amino acid commonly used can be exemplified as the amino acid, and arginine, glutamine, aspartic acid, alanine, proline and lysine can be use as a preferable amino acid.

The amino acid contains branched amino acid (BCAA), and leucine, isoleucine and valine can be exemplified as the branched amino acid. It is preferable to use two or more kinds of branched amino acids in combination, and in this case, the preferable mixing ratio is leucine:isoleucine:valine=3:2:2 (by weight, same in the following).

As soybean protein, for example, separated soybean protein, defatted soybean protein and soybean powder can be used.

As iron compound, for example, heme iron, iron gluconate and ferric chloride can be used.

As polyphenols, for example, proanthocyanidin, anthocyanine and oleuropein can be used.

Maca extract powder and hercampure extract powder can be prepared by a conventional method, and these are already on the market and it is preferable to use such commercially available goods.

In the invention, there is no particular limitation on the blending ratio of carnosine and/or anserine to the above-mentioned material for improving motor performance provided that it is possible to obtain synergistic effect, but the blending ratio of carnosine and/or anserine to the material for improving motor performance is adjusted to about 1:0.5 to 10, preferably 1:1.0 to 5.0, more preferably 1:1.5 to 4.0.

The ingested amount of the food for improving motor function of the invention can be properly adjusted according to the desired levels of improvements of exercise function and stamina, levels of fatigue, weight and age. Generally, it is 30 to 500 mg/kg by weight, preferably 50 to 300 mg/kg by weight, more preferably 100 to 200 mg/kg by weight (carnosine and anserine contents are of purity conversion). It is preferable to take such amount of the food once or separately several times per day.

The food for improving motor function of the invention is ingested by humans solely or in a variety of forms such as solid form food, food and drink.

As the solid form food, each of the above-mentioned components is mixed with physiologically permitted components necessary for formulation such as additives (e.g. carrier, excipient, diluent, etc.) and formulated to an appropriate form. As such forms, tablets, powder, granule, and capsules can be exemplified.

Also, as the food and drink, for example, the above-mentioned components are added to drinks (e.g. health drink, milk, coffee, black tea, green tea, juice, etc.), cakes (biscuit, cookie, candy, snack, lemon pop, etc.), seasonings (sauce, etc.), meats (ham, sausage, etc.), fish meats (boiled fish paste, chikuwa (a kind of fish paste), etc.) and dairy products (butter, cheese, etc.).

Such food and drink can be prepared by a conventional method except that the effective components are added at an appropriate step in the stage of preparation. Also, it is preferable to add ordinary additives as needed, and as available such additives are, for example, vitamins (e.g. vitamin C, vitamin A, vitamin E, etc.), minerals (e.g. zinc, copper, manganese, etc.), hormones, physiologically active substance, sweetening, sour, antioxidant, spice, salt, excipient, coloring material.

Further ordinary components in this field can be added to the food for improving motor function of the invention as needed.

The food for improving motor function of the invention is effective for realizing the physical performance improvement, stamina improvement and recovery from fatigue of humans. Also, the food for improving motor function of the invention is effective for realizing the physical performance improvement, stamina improvement and recovery from fatigue of various animals as well as humans. In this specification, animals stand for mammals other than humans for the sake of convenience, and as such mammals, domestic animals such as horse, cow, pig, and sheep, and companion animals such as dog, cat, etc. can be exemplified.

### INDUSTRIAL APPLICABILITY

The food for improving motor function of the invention contains carnosine and/or anserine and the material for improving motor performance as effective components, and due to the synergistic effect of these components, it is possible to realize the improvements of physical performance, stamina and recovery from fatigue of humans or mammal. Further, all the effective components of the food for improving motor function of the invention are naturally occurring components, therefore, the food has a characteristic of high safety.

### EXAMPLES

The present invention will be specifically explained in detail with Examples, but the invention is not limited by these Examples.

### Manufacturing Example 1

Manufacture of Chicken Extract Powder Containing Carnosine and Anserine

Chicken extract powder containing carnosine and anserine was manufactured by the following processes. Chicken was sliced by a meat grinder, and after adding hot water by 1.5 times the weight of meat, citric acid was added by 0.2% as against the weight of meat to adjust to pH5.5. An extraction was carried out at 90° C. for four hours, and insoluble matters of over 100 mesh were removed by filtering to obtain an extracted solution. Papain was added thereto by 0.06% as against the extracted solution, and after reacting at 50° C. for one hour, enzyme was deactivated by heating at 90° C. for 15 minutes. Subsequently, dry powder of less than 50 mesh was obtained through freeze drying and grinding. Chicken extract powder thus obtained was used in the following tests.

The chicken extract powder contains carnosine by 5% and anserine by 10%.

### Example 1

Improvement of Stamina By Administering the Chicken Extract Powder

The test solution was given to mice to check for the influence on the improvement of stamina. Five-week-old male ddY mice (purchased from Japan SLC Co. Ltd.) were raised, letting them be free to take solid feed (CE-2, Japan Clare Co. Ltd., Tokyo) and water (10 mice per group).

Further, they were trained by swimming every other day for 20 minutes each totaling two times in a running water pool (velocity at center: 15.6 cm/sec.). After one week of preparatory raising and swim training, they were allowed to freely take ordinary solid feed and water. At the same time, a mixture solution of the chicken extract powder 30 mg/ml +creatine 10 mg/ml was orally given to them by the amount of 10 ml/kg once a day for seven days. On the day just before start of giving the test solution and on the 7th day after start of giving the test solution, the experimental animals were let to swim until exhaustion in the pool (velocity at center: 23.6 cm/sec.) to measure the swimming duration and to obtain the elongation of the swimming duration in seven days. The results are shown as relative values (%) when the average value of elongation percentage of Comparative Example 21 (control) is 100.

### Example 2

The same treatment of Example 1 was carried out except that a mixture solution of the chicken extract power 30 mg/ml+arginine 10 mg/ml was given to the mice.

### Example 3

The same treatment of Example 1 was carried out except that a mixture solution of the chicken extract power 30 mg/ml+branched amino acid (leucine:isoleucine:valine=3:2:2) 10 mg/ml was given to the mice.

### Example 4

The same treatment of Example 1 was carried out except that a mixture solution of the chicken extract power 30 mg/ml+whey protein 10 mg/ml was given to the mice.

### Example 5

The same treatment of Example 1 was carried out except that a mixture solution of the chicken extract power 30 mg/ml+separated soybean protein 10 mg/ml was given to the mice.

### Example 6

The same treatment of Example 1 was carried out except that a mixture solution of the chicken extract power 30 mg/ml+caffeine 10 mg/ml was given to the mice.

### Example 7

The same treatment of Example 1 was carried out except that a mixture solution of the chicken extract power 30 mg/ml+citric acid 10 mg/ml was given to the mice.

### Example 8

The same treatment of Example 1 was carried out except that a mixture solution of the chicken extract power 30 mg/ml+carnitine 10 mg/ml was given to the mice.

### Example 9

The same treatment of Example 1 was carried out except that a mixture solution of the chicken extract power 30 mg/ml+thioctic acid 10 mg/ml was given to the mice.

### Example 10

The same treatment of Example 1 was carried out except that a mixture solution of the chicken extract power 30 mg/ml+heme iron 10 mg/ml was given to the mice.

### Example 11

The same treatment of Example 1 was carried out except that a mixture solution of the chicken extract power 30 mg/ml+proanthocyanidin 10 mg/ml was given to the mice.

### Example 12

The same treatment of Example 1 was carried out except that a mixture solution of the chicken extract power 30 mg/ml+anthocyanine 10 mg/ml was given to the mice.

### Example 13

The same treatment of Example 1 was carried out except that a mixture solution of the chicken extract power 30 mg/ml+oleuropein 10 mg/ml was given to the mice.

### Example 14

The same treatment of Example 1 was carried out except that a mixture solution of the chicken extract power 30 mg/ml+ubiquinone (CoQ10) 10 mg/ml was given to the mice.

### Example 15

The same treatment of Example 1 was carried out except that a mixture solution of the chicken extract power 30 mg/ml+tocotrienol 10 mg/ml was given to the mice.

### Example 16

The same treatment of Example 1 was carried out except that a mixture solution of the chicken extract power 30 mg/ml+liquamen 10 mg/ml was given to the mice.

### Example 17

The same treatment of Example 1 was carried out except that a mixture solution of the chicken extract power 30 mg/ml+maca extract powder 10 mg/ml was given to the mice.

### Example 18

The same treatment of Example 1 was carried out except that a mixture solution of the chicken extract power 30 mg/ml+hercampure extract powder 10 mg/ml was given to the mice.

### Example 19

The same treatment of Example 1 was carried out except that a mixture solution of the chicken extract power 30 mg/ml+banasulin 10 mg/ml was given to the mice.

### Comparative Example 1

The same treatment of Example 1 was carried out except that a solution of the creatine 10 mg/ml was given to the mice.

### Comparative Example 2

The same treatment of Comparative Example 1 was carried out except that a solution of arginine 10 mg/ml was given to the mice.

### Comparative Example 3

The same treatment of Comparative Example 1 was carried out except that a solution of branched amino acid (leucine:isoleucine:valine=3:2:2) 10 mg/ml was given to the mice.

### Comparative Example 4

The same treatment of Comparative Example 1 was carried out except that a solution of whey protein 10 mg/ml was given to the mice.

### Comparative Example 5

The same treatment of Comparative Example 1 was carried out except that a solution of separated soybean protein 10 mg/ml was given to the mice.

### Comparative Example 6

The same treatment of Comparative Example 1 was carried out except that a solution of the caffeine 10 mg/ml was given to the mice.

### Comparative Example 7

The same treatment of Comparative Example 1 was carried out except that a solution of the citric acid 10 mg/ml was given to the mice.

### Comparative Example 8

The same treatment of Comparative Example 1 was carried out except that a solution of the carnitine 10 mg/ml was given to the mice.

### Comparative Example 9

The same treatment of Comparative Example 1 was carried out except that a solution of thioctic acid 10 mg/ml was given to the mice.

### Comparative Example 10

The same treatment of Comparative Example 1 was carried out except that a solution of heme iron 10 mg/ml was given to the mice.

### Comparative Example 11

The same treatment of Comparative Example 1 was carried out except that a solution of proanthocyanidin 10 mg/ml was given to the mice.

### Comparative Example 12

The same treatment of Comparative Example 1 was carried out except that a solution of antocyanine 10 mg/ml was given to the mice.

### Comparative Example 13

The same treatment of Comparative Example 1 was carried out except that a solution of oleuropein 10 mg/ml was given to the mice.

### Comparative Example 14

The same treatment of Comparative Example 1 was carried out except that a solution of ubiquinone (CoQ10) 10 mg/ml was given to the mice.

### Comparative Example 15

The same treatment of Comparative Example 1 was carried out except that a solution of tocotrienol 10 mg/ml was given to the mice.

### Comparative Example 16

The same treatment of Comparative Example 1 was carried out except that a solution of liquamen 10 mg/ml was given to the mice.

### Comparative Example 17

The same treatment of Comparative Example 1 was carried out except that a solution of maca extract powder 10 mg/ml was given to the mice.

### Comparative Example 18

The same treatment of Comparative Example 1 was carried out except that a solution of hercampure extract powder 10 mg/ml was given to the mice.

### Comparative Example 19

The same treatment of Comparative Example 1 was carried out except that a solution of banasulin 10 mg/ml was given to the mice.

### Comparative Example 20

The same treatment of Comparative Example 1 was carried out except that a solution of the chicken extract powder 30 mg/ml was given to the mice.

### Comparative Example 21 (Control)

The same treatment of Comparative Example 1 was carried out except that only water instead of the solution was given to the mice.

The results of the above tests are shown in Table 1 below.

TABLE 1
Stamina improving effects (10 animals per group)

| Elongation of | | | Without | | With |
|---|---|---|---|---|---|
| swimming | Material for | | chicken | | chicken |
| duration | improving motor | | extract | | extract |
| (%) | performance | | powder | | powder |
| Creatine | Material for motor | Comparative | 110 | Example 1 | 190 |
| | energy source | Example 1 | | | |
| Arginine | Material for motor | Comparative | 110 | Example 2 | 160 |
| | energy source | Example 2 | | | |
| Branched | Material for motor | Comparative | 110 | Example 3 | 180 |
| amino acid | energy source | Example 3 | | | |
| Whey protein | Material for motor | Comparative | 120 | Example 4 | 160 |
| | energy source | Example 4 | | | |
| Separated soy- | Material for motor | Comparative | 120 | Example 5 | 160 |
| bean protein | energy source | Example 5 | | | |
| Caffeine | Material for | Comparative | 110 | Example 6 | 190 |
| | Promoting conversion | Example 6 | | | |
| | into energy | | | | |
| Citric acid | Material for | Comparative | 110 | Example 7 | 180 |
| | promoting conversion | Example 7 | | | |
| | into energy | | | | |
| Carnitine | Material for | Comparative | 110 | Example 8 | 180 |
| | promoting conversion | Example 8 | | | |
| | into energy | | | | |
| Thioctic acid | Material for | Comparative | 110 | Example 9 | 180 |
| | promoting conversion | Example 9 | | | |
| | into energy | | | | |
| Heme iron | Material for | Comparative | 110 | Example 10 | 170 |
| | promoting conversion | Example 10 | | | |
| | into energy | | | | |
| Proantocyanidin | Antioxidant | Comparative | 110 | Example 11 | 170 |
| | | Example 11 | | | |
| Antocyanine | Antioxidant | Comparative | 120 | Example 12 | 170 |
| | | Example 12 | | | |
| Oleuropein | Antioxidant | Comparative | 120 | Example 13 | 170 |
| | | Example 13 | | | |
| Ubiquinone | Antioxidant | Comparative | 110 | Example 14 | 170 |
| (CoQ10) | | Example 14 | | | |
| Tocotrienol | Antioxidant | Comparative | 120 | Example 15 | 170 |
| | | Example 15 | | | |
| Liquamen | Antioxidant | Comparative | 120 | Example 16 | 190 |
| | | Example 16 | | | |
| Maca extract | Antioxidant | Comparative | 110 | Example 17 | 180 |
| powder | | Example 17 | | | |
| Hercampure | Antioxidant | Comparative | 120 | Example 18 | 180 |
| extract powder | | Example 18 | | | |
| Banasulin | Antioxidant | Comparative | 120 | Example 19 | 170 |
| | | Example 19 | | | |
| None | — | Comparative | 100 | Example 20 | 120 |
| | | Example 21 | | | |

In Table 1, “none” stands for non-addition. The elongation of swimming duration (%) is shown as relative values when the average value of Comparative Example 21 is 100.

As shown in Table 1, when the stamina improving effect of Comparative Example 21 (control) is 100%, the improving effect is only 120% in the group with only the chicken extract powder given. In the case of giving a mixture of the chicken extract powder and the material for improving motor performance (i.e. materials for motor energy source, materials for promoting conversion into energy and antioxidant), the stamina improving effect is as high as 160 to 190% of control value, showing that remarkable synergistic effect can be obtained by using the chicken extract powder and the material for improving motor performance in combination. In this case, there is no difference in increase of the weight between both groups.

The results thus obtained show that a mixture of the chicken extract powder +the material for improving motor performance is able to significantly enhance the stamina in the swimming test. Accordingly, it has been found that the food for improving motor function of the invention brings about an excellent stamina improving effect.

### Examples 20 to 22

The same treatment of Example 1 was carried out except that a solution of the chicken extract powder 30 mg/ml+creatine 10 mg/ml+arginine 10 g/ml (Example 20); the chicken extract powder 30 mg/ml+creatine 10 mg/ml+carnitine 10 mg/ml (Example 21) or the chicken extract powder 30 mg/ml+carnitine 10 mg/ml+antocyanine 10 mg/ml (Example 22) was used for the purpose of obtaining the elongation of swimming duration after seven days, and the results are shown as relative values (%) when the average value of elongation percentage of Comparative Example 21 (control) is 100.

As a result, the elongation percentage of swimming duration is as follows:
Example 20: 250%
Example 21: 300%
Example 22: 290%

### Manufacturing Example 1

Manufacture of Capsule Food

(1) A mixture (2 g) of three weight part of the chicken extract powder and one weight part of creatine was filled into a hard capsule by a conventional method, thereby manufacturing capsule food.

(2) A mixture (2 g) of three weight part of the chicken extract powder and one weight part of caffeine was filled into a hard capsule by a conventional method, thereby manufacturing capsule food.

(3) A mixture (2 g) of three weight part of the chicken extract powder and one weight part of oleuropein was filled into a hard capsule by a conventional method, thereby manufacturing capsule food.

### Manufacturing Example 2

Manufacture of Fruit Drink

Citric acid (0.2 part), orange juice (35 part) and sugar (5 part) were dissolved in water (29.8 part) with heating, to which a solution (30 ml) of a mixture of the chicken extract powder (300 mg/ml)+carnitine (100 mg/ml) was added, and after cooling, the solution was filled into a container and sterilized at 85° C. for 30 minutes, thereby manufacturing a fruit drink.

1. Food for improving motor function containing carnosine and/or anserine, and a material for improving motor performance.

2. Food for improving motor function of claim 1, wherein the material for improving motor performance is a material for motor energy source, a material for promoting conversion into energy and/or an antioxidant.

3. Food for improving motor function of claim 2, wherein the material for motor energy source is creatine, amino acid, whey protein and/or soybean protein; the material for promoting conversion into energy is caffeine, citric acid, camitine, thioctic acid and/or iron compound; and the antioxidant is polyphenol, ubiquinone (CoQ10), tocotrienol, liquamen, maca extract powder, hercampure extract powder and/or banasulin.

4. Food for improving motor function of any one of claims 1 to 3, wherein the food contains carnosine and/or anserine and the material for improving motor performance at a ratio of 1:0.5 to 10 (by weight).

5. Food for improving motor function of claim 1, wherein camosine and/or anserine is blended as chicken extract or its powder.

6. Food for improving motor function of claim 1, wherein the form of the food is solid food, drinks, cakes, seasonings, meat product, fish product or dairy product.

7. A method for improving motor function, comprising administering the food of claim 1 to a mammal in need thereof.

8. The method according to claim 7, wherein said mammal is a human.
